(19) United States
(12) Patent Application Publication
(10) Pub. No.: US 2022/0218438 A1
(43) Pub. Date: Jul. 14, 2022

(54) CREATING THREE-DIMENSIONAL (3D) ANIMATION

(71) Applicant: OrthoSnap Corp., Westbury, NY (US)

(72) Inventors: Yan Pogorelsky, Westbury, NY (US); Michael Yoon, Westbury, NY (US)

(21) Appl. No.: 17/148,902

(22) Filed: Jan. 14, 2021

(51) Int. Cl. A61C 7/00; G16H 20/00; G06T 13/20; G06T 17/00

(52) U.S. Cl. CPC A61C 7/002; G06T 17/00; G06T 13/20; G16H 20/00

(57) ABSTRACT

Provided herein is a system for creating a 3D animation file of a patient's dental treatment plan. The system may provide a model of a perfect or final state of teeth after treatment. The system may capture changes to the model which result in a patient's current state of the teeth. The system may then generate an animation, in reverse, which morphs the current state of the patient's teeth into the final state. The example embodiments provide an animation file which can be provided to the patient via telemedicine rather than an in-person visit.

### BACKGROUND

Orthodontic aligners are an alternative to traditional metal braces. Orthodontic aligners consist of removable trays, often made of clear plastic material, which fit over the teeth. Because the aligners are typically made of a clear plastic material, the aligners are considered invisible. A typical use case requires a set of trays which are used in sequence (e.g., for one to two weeks at a time) and which slowly move the teeth. The trays can be designed using state-of-the art techniques based on models and/or images of the teeth. Often, the trays are generated using three-dimensional (3D) printing. The aligners work because slight changes provided by the sequence of trays gradually shifts the teeth to a desired place.

Before a patient agrees to a treatment plan, they often require an in-person visit with a dental professional (dentist, orthodontist, etc.) to discuss treatment options. During this visit, the medical professional may capture images/scans of the patient's teeth and use them as a basis for creating the aligners. This also provides the patient an opportunity to ask questions about the procedure, the effects, the timeline, and the like. However, not all patients are capable of visiting a dental professional in-person. In fact, many patients now prefer telemedicine over in-person benefits. Therefore, what is needed is a way to engage patients in their treatment plans through remote means.

### BRIEF DESCRIPTION OF THE DRAWINGS

Features and advantages of the example embodiments, and the manner in which the same are accomplished, will become more readily apparent with reference to the following detailed description taken in conjunction with the accompanying drawings.

FIG. 1A is a diagram illustrating a predefined image being extracted from a library in accordance with an example embodiment.

FIG. 1B is a diagram illustrating a layout of a user interface for creating 3D animation in accordance with an example embodiment.

FIGS. 2A-2D are diagrams illustrating processes of modifying positions of teeth via the user interface in accordance with an example embodiment.

FIG. 3 is a diagram illustrating a process of creating a 3D animation in accordance with an example embodiment.

FIG. 4 is a diagram illustrating of a method of creating a 3D animation in accordance with an example embodiment.

FIG. 5 is a diagram illustrating a computing system for use in the examples herein in accordance with an example embodiment.

Throughout the drawings and the detailed description, unless otherwise described, the same drawing reference numerals will be understood to refer to the same elements, features, and structures. The relative size and depiction of these elements may be exaggerated or adjusted for clarity, illustration, and/or convenience.

### DETAILED DESCRIPTION

In the following description, specific details are set forth in order to provide a thorough understanding of the various example embodiments. It should be appreciated that various modifications to the embodiments will be readily apparent to those skilled in the art, and the generic principles defined herein may be applied to other embodiments and applications without departing from the spirit and scope of the disclosure. Moreover, in the following description, numerous details are set forth for the purpose of explanation. However, one of ordinary skill in the art should understand that embodiments may be practiced without the use of these specific details. In other instances, well-known structures and processes are not shown or described in order not to obscure the description with unnecessary detail. Thus, the present disclosure is not intended to be limited to the embodiments shown but is to be accorded the widest scope consistent with the principles and features disclosed herein.

The example embodiments are directed to a system which can generate a three-dimensional (3D) animation representing a treatment (e.g., orthodontic aligners, etc.) that is to be performed on a set of teeth. For example, the 3D animation may be an animated morphology which illustrates an animation between a patient's teeth prior to the treatment and what the patient's teeth will look like after the treatment. To generate the animation, a user may select a dental model (animation model) which corresponds to a final state of teeth after treatment. The dental model may be an animated image that is selected from a library of images which each represent a different dental model of ideal teeth after treatment. Here, the user may select the dental model based on a photo or other information provided by the patient so the dental model most closely matches the patient's dentition.

The user may enter commands via the user interface to change the position, rotation, tilt, angle, and the like, of teeth within the dental model of the final state of the teeth after treatment. For example, the user may modify the teeth to match a patient's actual teeth arrangement before treatment (e.g., as they are now). The system may provide a cursor which enables the user to move and select individual teeth. The system may also provide different commands to allow the user to manipulate the teeth positioning and angle. Also, the system may allow the user to remove one or more teeth from the dental model. In some embodiments, the user interface may display a photo of the user's actual teeth next to the dental model to allow the user to make changes to the dental model based on actual photos/images of the patient's teeth.

When the user is done, the user may enter a command to create a 3D animation of the change in the patient's teeth (positioning, angle, etc.). However, rather than playing the animation in the order in which the changes/modifications are made via the user interface, the system can transform the state of the teeth in reverse (i.e., from the final state of the teeth created by the last modification to the perfect teeth (selected animation image) representing what the patient's teeth will look like after treatment. The result is an animated video which morphs from the patient's current teeth to what the patient's teeth will look like after treatment.

By playing the animation in reverse, the transformation starts with the patient's current state (i.e., what the patient's teeth currently look like) and finishes with the final state of the patient's teeth after treatment (i.e., what the patient's teeth will look like). Thus, a patient can be provided a video which presents an estimated change in their teeth as a result of a proposed course of treatment which is yet to be performed. Accordingly, a patient can make a better informed decision on whether the proposed treatment is beneficial, and without having to visit a dentist's office to perform physical scans. That is, the present embodiments lend themselves to the use of telemedicine because all of the steps can be performed remotely by a dentist or technician who is not in physical presence of the patient, but simply has a photo of the patient's teeth . Further, the system can create the animation in reverse from the actual state of the patient's teeth to the final state (selected dental model before modifications by the user). In this example, the animation may generate a visualization in which the initial state of the patient's teeth morphs into the final state of the dental model thereby showing the patient how their teeth will change as a result of the treatment.

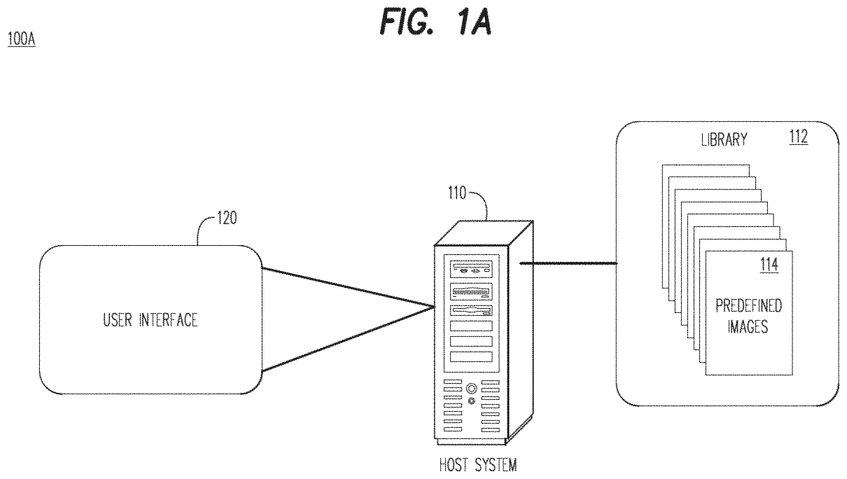

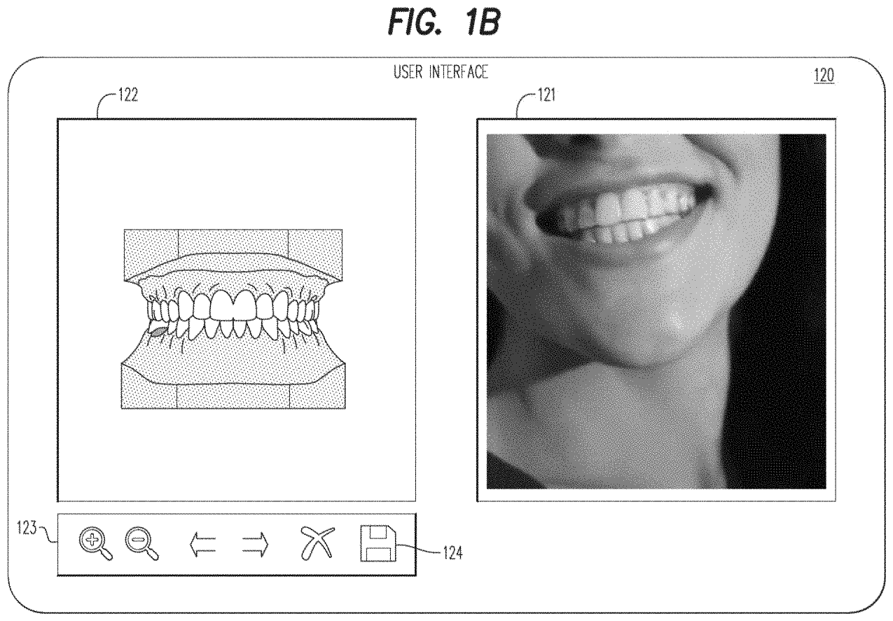

FIG. 1A illustrates a process of a predefined image being extracted from a library 112 in accordance with an example embodiment, and FIG. 1B illustrates a layout of a user interface 120 for creating 3D animation in accordance with an example embodiment. Referring to FIG. 1A, a host system 110 may host an animation tool (e.g., software application, service, program, etc.) which includes instructions/program code for creating animation from captured images. In this example, the host system 110 may be a user computing device (e.g., desktop computer, laptop, tablet, etc.), a network computer (e.g., on-premises server, web server, cloud platform, etc.), and the like. A user may execute the animation software locally on the host system 110 while accessing the host system locally (e.g., via input devices not shown, etc.), remotely (e.g., via an external device connected to the host system 110 via a network, Internet, etc.), and the like.

The host system 110 may output or otherwise display a user interface 120 (which is further shown in FIG. 1B) which can be manipulated by a user via input commands such as mouse clicks, touch inputs, keyboard commands, cursor scrolls, and the like. The host system 110 also stores a library 112 of predefined images 114 of teeth. The predefined images 114 are not representative of a particular user's teeth but rather animation models of what teeth will look like after a treatment plan has been performed. In other words, the predefined images 114 may be animation templates or models of “perfect teeth” or “fixed teeth” that are not an actual image of a person's teeth, but rather an ideal model of what the teeth will look like after treatment. For example, the library 112 may include a small set (e.g., 5, 10, 15, 25, 50, etc.) of predefined images 114 that can be used to match with a user's actual teeth.

Referring to FIG. 1B, the user interface 120 includes a photo window 121 for displaying an image of an actual user's teeth, smile, jaw, etc. For example, a user may upload a digital image (e.g., a photograph, a picture, etc.) of a patient's teeth which is populated by the software inside the photo window 121 of the user interface 120. Here, the image of the patient's teeth may represent what the patient's teeth look like before the treatment plan has been performed. The user interface 120 also includes an animation window 122 which is displayed adjacent to the photo window 121 which includes the photograph of the patient's teeth. Here, the animation window 122 may display the predefined images 114 from the library 112. The predefined images 114 may be still three-dimensional (3D) models which are configured to be animated in 3D

The user may use controls (e.g., the arrows, etc.) within a control panel 123 of the user interface 120 to scroll through the different predefined images within the animation window 122. Here, the predefined images are animation images and not photographs. Each time the user selects one of the arrows, the software may close a currently displayed predefined image and pull up a next predefined image. This process may be repeated in a loop in either direction such that the sequence of images are pulled up one at a time, in a sequence, and looping around from end to start. For example, if there are 10 predefined images, the user may scroll from a first image to a tenth image via the animation window 122 by selecting the arrow a number of different times until reaching the 10^{th} image. Furthermore, when the user reaches the 10^{th} predefined image, upon selection of the next button/scroll command, the software may loop back around to the first predefined image.

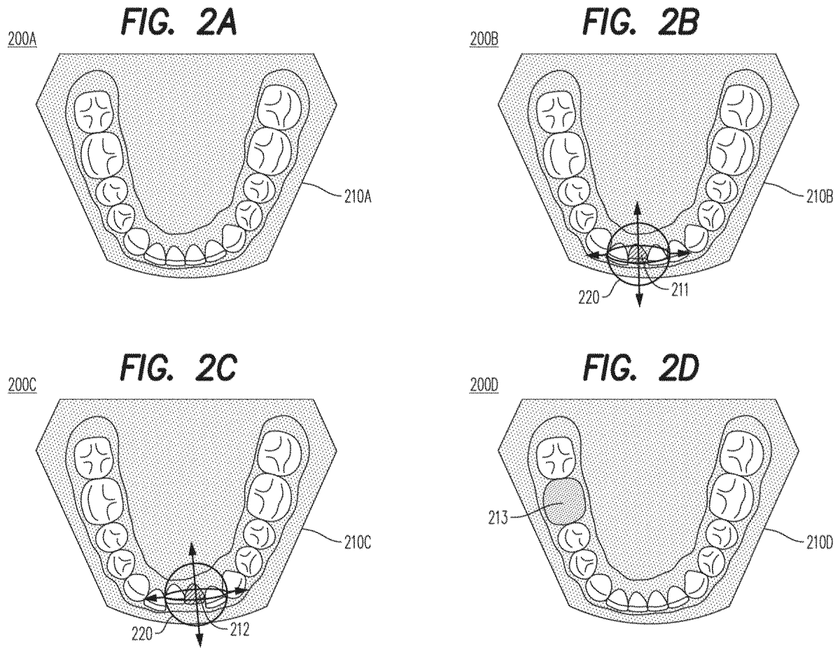

The user may continue to scroll through the predefined images via the animation window 122 until the user finds a predefined image (i.e., a still animation) that most closely matches the patient's teeth in the photograph in photo window 121. Here, the user is trying to match a size, shape, etc. of the patient's mouth with a predefined image 114, and not what the teeth currently look like. In other words, the predefined images 114 represent what an ideal set of teeth look like. The user is trying to match a style, shape, size, etc. of the patient's teeth with a predefined image 114 of ideal teeth, but not the actual teeth positions, rotations, etc. Once the user has matched a predefined image displayed within the animation window 122 to a photo of a patient's teeth in the photo window 121, the user may manipulate the predefined image to create an animation as further described in the examples of FIGS. 2A-2D.

FIGS. 2A-2D illustrate processes of modifying positions of teeth within a 3D image model in accordance with an example embodiment. For example, the modifications in the examples of FIGS. 2A-2D may be performed by a user making selections via the control panel 123 of the user interface 120 in FIG. 1B. Here, the 3D image model may be displayed within the animation window 122 while the user is viewing the photo of the patient's teeth in the photo window 121 of the user interface.

FIG. 2A illustrates a process 200A of initially displaying a selected 3D image model 210A of teeth. Here, the 3D image model may be selected from the predefined images 114 stored in the library 112 shown in FIG. 1A. The 3D image model 210A does not represent a current state of a patient's teeth but rather an ideal state of what the teeth will look like after treatment. Although only the bottom teeth are shown in the 3D image model 210A, it should be appreciated that both the top and bottom sections of teeth may be manipulated. Furthermore, the view of the teeth shown in FIG. 2A is from above, but it should also be appreciated that the user may view and manipulate the teeth at different angles. For example, the 3D image model 210 may be rotated in any direction (e.g., like a gimbal, etc.) which allows the orientation of the 3D image model 210A to be rotated around in any direction while maintaining a position of the predefined image in the center or other predefined area of the screen.

As shown in process 200B of FIG. 2B, the user may move a cursor 220 to a tooth 211 and select the tooth 211 for manipulation. At this point, although not shown, the user may have various interactive options to modify the selected tooth 211. For example, the user may twist, rotate, push in, pull out, move up, move down, etc. the tooth 211 with respect to the other teeth within the 3D image model 210A to create a modified 3D image model 210B. In particular, in the example of FIG. 2B, the tooth 211 is pushed inward towards the back of the mouth. The cursor 220 may include a gimbal like structure which provides notice that the tooth can be twisted or otherwise rotated in any direction, and also moved in any direction within the 3D image model.

As shown in process 200C of FIG. 2C, the user may select another tooth 212 for manipulation using the cursor 220. Here, the user may push the tooth 212 inward similar to the modification made to the tooth 211 in the modified 3D image model 210B of FIG. 2B, resulting in another modified 3D image model 210C. The user may also perform other manipulations to the teeth, for example, pulling outward, rotating in place, bending, tilting, etc. Furthermore, as shown in process 200D of FIG. 2D, the user may remove any of the teeth (e.g., tooth 213). The result is another modified 3D image model 210D.

While only a handful of manipulations are shown in the examples of FIGS. 2B-2D, it should be appreciated that dozens of manipulations may be made to the originally selected 3D image model 210A to generate a more accurate representation of the current state of a patient's teeth. When the user has reached a point where they believe the manipulated 3D image model represents the actual patient's teeth, the user may create an animation. For example, the user may select a button 124 shown in FIG. 1B to create a reverse transformation of the manipulations made to the selected image model 210A.

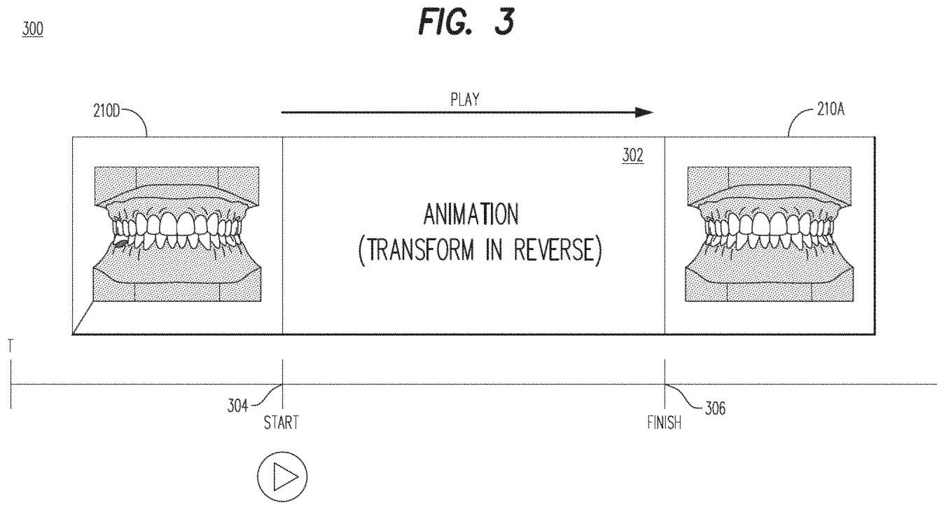

FIG. 3 illustrates a process 300 of creating a 3D animation in accordance with an example embodiment. For example, the software may capture or otherwise store still images of the 3D image model as it is transformed via the user interface. The software may use the still images of the 3D image model to create and play the animation (manipulation of image data to appear as moving images in 3D). In particular, the system may play the animation in reverse from the final state of the teeth in the 3D image model 210D with all of the modifications made by the user via the user interface 120 to the initial state of the teeth in the predefined 3D image model 210A selected by the user. By playing the animation in reverse, the transformation starts with the patient's current state (i.e., what the patient's teeth currently look like) and finishes with the final state of the patient's teeth after treatment (i.e., what the patient's teeth will look like).

As shown in FIG. 3, a start 304 of the animation may be the final state of the patient's teeth in modified 3D image model 210D which transforms into the initially selected predefined 3D image model 210A representing what the patient's teeth will look like by an end 306 of the animation. The playing time between the start 304 and the finish 306 may include visual 3D transformations (animations) to the teeth which make the teeth appear to move from their current state into their expected state. The result is an animated video that can visualize the changes that will be made to the user's teeth through 3D animation.

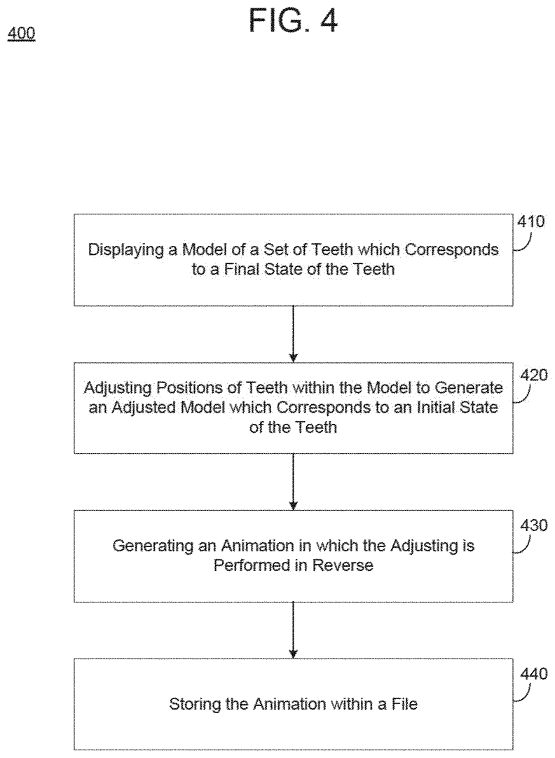

FIG. 4 illustrates a method 400 of creating a 3D animation in accordance with an example embodiment. For example, the method 400 may be performed by a user device, a web server, a cloud platform, a combination of devices/nodes, or the like. Referring to FIG. 4, in 410, the method may include displaying, via a user interface, a model of a set of teeth which corresponds to a final state of the set of teeth. For example, the model may be selected from among a plurality of models of different final states of teeth after a treatment procedure is performed. Here, the models are not the actual patient's teeth but standard models which could approximate a patient's final state after a treatment procedure is performed.

In some embodiments, the method may further include selecting the model of the set of teeth from among a plurality of predefined models representing a plurality of final states, respectively. In some embodiments, the displaying may include displaying the model of the set of teeth via a first window of the user interface and displaying a digital image in a second window of the user interface.

In 420, the method may include adjusting positions of a plurality of teeth of the model of the set of teeth based on inputs detected via the user interface to generate an adjusted model which corresponds to an initial state of the set of teeth. For example, the adjusting of the positions may include one or more of pushing a position of a tooth inward, pushing a position of a tooth outward, rotating a position of a tooth, and removing a tooth. Here, a user may control a cursor which has a multi-gimbal functionality that allows a selected tooth to be rotated around different axes in three dimensions. Furthermore, the system may allow the user to push a tooth inward with respect to the rest of the teeth, pull a tooth outward, pull a tooth downward, pull a tooth upward, move a tooth, delete a tooth, and the like.

In 430, the method may include generating an animation in which the adjusting of the positions of the plurality of teeth are performed in reverse, and in 440, the method may include storing the animation within a file. In some embodiments, the generating may include recording an animation of the visualization of the final state of the set of teeth morphing into the adjusted visualization of the initial state of the set of teeth. In some embodiments, the generating may further include configuring the recorded animation to play in reverse prior to generate the reverse animation. For example, the animation starts with the adjusted model and undoes the adjusted positions in reverse to return the adjusted model to the model of the final state of the set of teeth. In some embodiments, the generating may include generating a three-dimensional animation in which the adjusting of the set of teeth is animated in three dimensions.

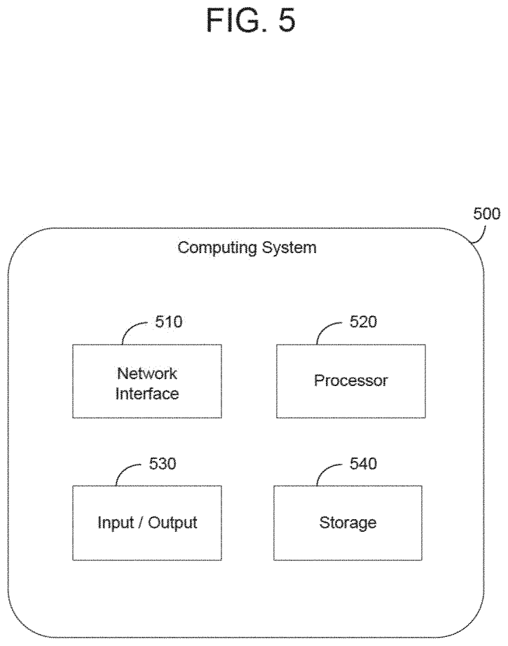

FIG. 5 illustrates a computing system 500 that may be used in any of the methods and processes described herein, in accordance with an example embodiment. For example, the computing system 500 may be a database node, a server, a cloud platform, or the like. In some embodiments, the computing system 500 may be distributed across multiple computing devices such as multiple database nodes. Referring to FIG. 5, the computing system 500 includes a network interface 510, a processor 520, an input/output 530, and a storage device 540 such as an in-memory storage, and the like. Although not shown in FIG. 5, the computing system 500 may also include or be electronically connected to other components such as a display, an input unit(s), a receiver, a transmitter, a persistent disk, and the like. The processor 520 may control the other components of the computing system 500.

The network interface 510 may transmit and receive data over a network such as the Internet, a private network, a public network, an enterprise network, and the like. The network interface 510 may be a wireless interface, a wired interface, or a combination thereof. The processor 520 may include one or more processing devices each including one or more processing cores. In some examples, the processor 520 is a multicore processor or a plurality of multicore processors. Also, the processor 520 may be fixed or it may be reconfigurable. The input/output 530 may include an interface, a port, a cable, a bus, a board, a wire, and the like, for inputting and outputting data to and from the computing system 500. For example, data may be output to an embedded display of the computing system 500, an externally connected display, a display connected to the cloud, another device, and the like. The network interface 510, the input/output 530, the storage 540, or a combination thereof, may interact with applications executing on other devices.

The storage device 540 is not limited to a particular storage device and may include any known memory device such as RAM, ROM, hard disk, and the like, and may or may not be included within a database system, a cloud environment, a web server, or the like. The storage 540 may store software modules or other instructions which can be executed by the processor 520 to perform the method shown in FIG. 5. According to various embodiments, the storage 540 may include a data store that stores data in one or more formats such as a multidimensional data model, a plurality of tables, partitions and sub-partitions, and the like. The storage 540 may be used to store database records, items, entries, and the like.

According to various embodiments, the processor 520 may be configured to receive an identification of a measure of multidimensional data, generate a plurality of predictive data sets where each predictive data set comprises a different combination of dimension granularities used for aggregation, train a plurality of instances of a machine learning model based on the plurality of predictive data sets, respectively, and determine and output predictive performance values of the plurality of instances of the trained machine learning model. For example, the processor 520 may be configured to perform any of the functions, methods, operations, etc., described above with respect to FIGS. 2A-2E, 3, and 4.

As will be appreciated based on the foregoing specification, the above-described examples of the disclosure may be implemented using computer programming or engineering techniques including computer software, firmware, hardware or any combination or subset thereof. Any such resulting program, having computer-readable code, may be embodied or provided within one or more non-transitory computer-readable media, thereby making a computer program product, i.e., an article of manufacture, according to the discussed examples of the disclosure. For example, the non-transitory computer-readable media may be, but is not limited to, a fixed drive, diskette, optical disk, magnetic tape, flash memory, external drive, semiconductor memory such as read-only memory (ROM), random-access memory (RAM), and/or any other non-transitory transmitting and/or receiving medium such as the Internet, cloud storage, the Internet of Things (IoT), or other communication network or link. The article of manufacture containing the computer code may be made and/or used by executing the code directly from one medium, by copying the code from one medium to another medium, or by transmitting the code over a network.

The computer programs (also referred to as programs, software, software applications, “apps”, or code) may include machine instructions for a programmable processor, and may be implemented in a high-level procedural and/or object-oriented programming language, and/or in assembly/machine language. As used herein, the terms “machine-readable medium” and “computer-readable medium” refer to any computer program product, apparatus, cloud storage, internet of things, and/or device (e.g., magnetic discs, optical disks, memory, programmable logic devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal that may be used to provide machine instructions and/or any other kind of data to a programmable processor.

The above descriptions and illustrations of processes herein should not be considered to imply a fixed order for performing the process steps. Rather, the process steps may be performed in any order that is practicable, including simultaneous performance of at least some steps. Although the disclosure has been described in connection with specific examples, it should be understood that various changes, substitutions, and alterations apparent to those skilled in the art can be made to the disclosed embodiments without departing from the spirit and scope of the disclosure as set forth in the appended claims.

What is claimed is:

1. A computing system comprising:
a memory; and
a processor configured to
display, via a user interface, a model of a set of teeth which corresponds to a final state of the set of teeth,
adjust positions of a plurality of teeth of the model of the set of teeth based on inputs detected via the user interface to generate an adjusted model which corresponds to an initial state of the set of teeth,
generate an animation in which the adjusting of the positions of the plurality of teeth are performed in reverse, and
store the animation within a file in the memory.

2. The computing system of claim 1, wherein the processor is further configured to select the model of the set of teeth from among a plurality of predefined models representing a plurality of final states, respectively.

3. The computing system of claim 1, wherein the processor is configured to record an animation of the final state of the set of teeth morphing into the adjusted model of the initial state of the set of teeth.

4. The computing system of claim 3, wherein the processor configures the recorded animation to play in reverse prior to generate the reverse animation.

5. The computing system of claim 1, wherein the animation starts with the adjusted model and undoes the adjusted positions in reverse to return the adjusted model to the model of the final state of the set of teeth.

6. The computing system of claim 1, wherein the processor is configured to one or more of push a position of a tooth inward, push a position of a tooth outward, rotate a position of a tooth, and remove a tooth, during the adjusting.

7. The computing system of claim 1, wherein the processor is configured to generate a three-dimensional animation in which the adjusting of the set of teeth is animated in three dimensions.

8. The computing system of claim 1, wherein the model of the set of teeth via a first window of the user interface and displaying a digital image in a second window of the user interface.

9. A method comprising:
displaying, via a user interface, a model of a set of teeth which corresponds to a final state of the set of teeth;
adjusting positions of a plurality of teeth of the model of the set of teeth based on inputs detected via the user interface to generate an adjusted model which corresponds to an initial state of the set of teeth;
generating an animation in which the adjusting of the positions of the plurality of teeth are performed in reverse; and
storing the animation within a file.

10. The method of claim 9, further comprising selecting the model of the set of teeth from among a plurality of predefined models representing a plurality of final states, respectively.

11. The method of claim 9, wherein the generating comprises recording an animation of the visualization of the final state of the set of teeth morphing into the adjusted visualization of the initial state of the set of teeth.

12. The method of claim 11, wherein the generating further comprises configuring the recorded animation to play in reverse prior to generate the reverse animation.

13. The method of claim 9, wherein the animation starts with the adjusted model and undoes the adjusted positions in reverse to return the adjusted model to the model of the final state of the set of teeth.

14. The method of claim 9, wherein the adjusting of the positions comprises one or more of pushing a position of a tooth inward, pushing a position of a tooth outward, rotating a position of a tooth, and removing a tooth.

15. The method of claim 9, wherein the generating comprises generating a three-dimensional animation in which the adjusting of the set of teeth is animated in three dimensions.

16. The method of claim 9, wherein the displaying comprises displaying the model of the set of teeth via a first window of the user interface and displaying a digital image in a second window of the user interface.

17. A non-transitory computer-readable medium comprising instructions which when executed cause a computer to perform a method comprising:
displaying, via a user interface, a model of a set of teeth which corresponds to a final state of the set of teeth;
adjusting positions of a plurality of teeth of the model of the set of teeth based on inputs detected via the user interface to generate an adjusted model which corresponds to an initial state of the set of teeth;
generating an animation in which the adjusting of the positions of the plurality of teeth are performed in reverse; and
storing the animation within a file.

18. The non-transitory computer-readable medium of claim 17, wherein the method further comprises selecting the model of the set of teeth from among a plurality of predefined models representing a plurality of final states, respectively.

19. The non-transitory computer-readable medium of claim 17, wherein the generating comprises recording an animation of the visualization of the final state of the set of teeth morphing into the adjusted visualization of the initial state of the set of teeth.

20. The non-transitory computer-readable medium of claim 19, wherein the generating further comprises configuring the recorded animation to play in reverse prior to generate the reverse animation.
